(19) United States
(12) Patent Application Publication
(10) Pub. No.: US 2026/0025919 A1
(43) Pub. Date: Jan. 22, 2026

(54) ELONGATE CONDUCTOR ARRANGEMENT FOR ELECTRICAL CONNECTION TO A MEDICAL DEVICE, MEDICAL DEVICE AND METHOD FOR FABRICATING AN ELONGATE CONDUCTOR ARRANGEMENT

(71) Applicant: Biotronik SE & Co. KG, Berlin (DE)

(72) Inventors: Gernot Kolberg, Berlin (DE); Jens Rump, Berlin (DE); Dagmar Buchner, Berlin (DE); Joerg Feldmann, Berlin (DE)

(73) Assignee: Biotronik SE & Co. KG, Berlin (DE)

(21) Appl. No.: 18/998,592

(22) PCT Filed: Jul. 17, 2023
(86) PCT No.: PCT/EP2023/069742

§ 371 Date: Jan. 27, 2025

(30) Foreign Application Priority Data
Aug. 10, 2022 (EP) 22189721.8

(51) Int. Cl. H05K 1/03; A61N 1/05; H05K 1/02; H05K 3/22

(52) U.S. Cl. CPC H05K 1/032; A61N 1/05; H05K 1/0283; H05K 3/22; H05K 2201/0129; H05K 2201/0141; H05K 2201/09236; H05K 2203/1105

(57) ABSTRACT

An elongate conductor arrangement for electrical connection to a medical device is proposed, including an elongate sheet substrate and at least two conductor lines. Therein, the elongate sheet substrate comprises a support layer consisting of a LCP material. The conductor lines are arranged along a surface of the sheet substrate. The sheet substrate is twisted around a longitudinal axis of the sheet substrate into a spiral configuration. The conductor arrangement is easily bendable in various directions and allow a simple electrical connection to e.g. a circuitry of a medical device beneficially using LCP technology.

### CROSS-REFERENCE TO RELATED APPLICATIONS

This application is the United States National Phase under 35 U.S.C. § 371 of PCT International Patent Application No. PCT/EP2023/069742, filed on Jul. 17, 2023, which claims the benefit of European Patent Application No. 22189721.8, filed on Aug. 10, 2022, the disclosures of which are hereby incorporated by reference herein in their entireties.

### TECHNICAL FIELD

The present invention relates to an elongate conductor arrangement for electrical connection to a medical device. Furthermore, the present invention relates to a medical device including such elongate conductor arrangement and to a method for fabricating such elongate conductor arrangement.

### BACKGROUND

Various medical devices have been developed for monitoring physiological functions and/or providing medical treatments for a patient. For example, medical monitoring devices may measure various physiological parameters such as a body temperature, a pulse, a blood pressure, etc. Medical treatment devices such as pacemakers, cardioverters, defibrillators, neurostimulators, etc. may provide stimulations for actively influencing physiological parameters and/or physiological functions.

Such medical devices may require an elongate conductor arrangement for establishing an electrical connection to the medical device. For example, such conductor arrangement may provide an electrical connection between an electrode and a circuitry included in the medical device. The electrode may, for example, be implanted into the patient's body for sensing electrical potentials and/or for applying electrical voltages locally within the body. The medical device including its circuitries may be located at another position at or in the body. Accordingly, the elongate conductor arrangement has to electrically connect the electrode with the medical device, while at least part of this conductor arrangement being implanted into the body. Therein, due to physiological requirements and/or conditions within the body, the conductor arrangement is typically implanted such as to extend along a curved path throughout the body.

As will be discussed in more detail below, circuitries for modern medical devices may be embodied by beneficially using LCP technology. LCP (liquid crystal polymers) generally have advantageous physical characteristics such as superior mechanical loadability, high temperature tolerance, low chemical reactivity, etc. Accordingly, in LCP-technology, thin sheets of LCP may serve as a substrate for carrying conductor paths. Therein, LCP-technology may be used for fabricating implantable devices and products in a simple manner and/or with a high degree of automatization while enabling a high conductor density and/or a high degree of integration for various types of circuitries.

LCP substrates may be provided with a small thickness such as to be bendable. However, due to the sheet geometry, the LCP substrate is generally only bendable in one plane. Accordingly, in conventional approaches, LCP substrates carrying one or more conductor paths are difficult to be applied for providing an elongate conductor arrangement which is able to be bent in various directions, e.g., upon being implanted into a patient's body.

Thus, other concepts of elongate conductor arrangements such as wires, wire coils or ropes are generally to be used for establishing an electrical connection to a medical device including circuitries using LCP technology. However, a requirement of interconnecting conductor arrangements implemented as wires, wire coils or ropes, on the one side, and LCP-technology-based circuitries, on the other side, may limit options and advantages of the LCP technology and may therefore reduce its economic potential.

European Publication No. 3 292 885 A1 discloses an extendable electrode conductor arrangement and a medical implant. Therein, a zigzag-shaped or meander-shaped conductor path is applied on a supporting substrate. European Patent No. 3 181 188 B1 discloses an implantable electrode including a connector portion which is wound around a cylindrical core. Both approaches, LCP substrates may be used.

The present disclosure is directed toward overcoming one or more of the above-mentioned problems, though not necessarily limited to embodiments that do.

### SUMMARY

It is an object of the instant invention to provide an alternative elongate conductor arrangement for electrical connection to a medical device, wherein the elongate conductor arrangement is flexibly bendable, i.e., may be easily bent in various directions or planes, while being compatible with LCP technology. Furthermore, it may be an object of the instant invention to provide a medical device comprising such elongate conductor arrangement. Additionally, it may be an object of the instant invention to provide a method for fabricating such elongate conductor arrangement.

At least these objects may be achieved by the subject matter of the independent claims. Advantageous embodiments are disclosed in the dependent claims and the following specification as well as in the associated figures.

According to a first aspect of the present invention, an elongate conductor arrangement for electrical connection to a medical device is described to comprise an elongate sheet substrate and at least two conductor lines. Therein, the elongate sheet substrate comprises a support layer consisting of a LCP material. The conductor lines are arranged along a surface of the sheet substrate. The sheet substrate is twisted around a longitudinal axis of the sheet substrate into a spiral configuration.

This concept of a twisted sheet substrate for the conductor lines withstands the mechanical requirements for fatigue strength, possibly as the only conceivable concept in LCP technology. The main concern here is the bending load when forming the radius of the conductor arrangement. Because the twisted concept has a constantly changing preferred direction, a bending radius can be formed in any direction compared to a flat strip. A flat strip would only be able to be bent in one direction without stress. To differentiate: A coiled structure, on the other hand, only works well if the coiled filament has a round cross-section. The bend of a coil or helix is converted into a torsion of the single filament. If the filament is a flat strip, the torsion will cause the filament to tilt in the bent section. If the flat strip is very narrow or slim compared to the filament radius, or the bending can be strongly limited by other actions, the concept may probably be stably implemented. But for a space-saving solution, the space for tilting the filament would have to be provided by design. This would make the design unstable and large. If the space is not provided, constraining conditions will be created for the filament during bending, which would cause the filament to break under continuous load.

Regarding LCP (liquid crystal polymers)-one advantage here is that electrical and electronic components can be integrated very easily into LCP. LCP technology is also very good at forming mechanical structures (connector sleeves, ring electrodes, electrode surfaces). Very complex structures, such as electrode arrays, can be realized and electronic components can be integrated, so that a wide range of leads and electrodes is conceivable. The switch to LCP may be a quantum leap for electrode development because many new possibilities are created, e.g., multipolar electrodes that can be easily mapped with series processes: electrodes with complex electrode arrays: electrodes that contain electronic components, etc. However, the LCP concept only really makes sense if you stay in this technology. A mixed concept could have more disadvantages than advantages (expensive, large). If, for example, the LCP connector sleeves first have to be transferred to ropes or coils, large and expensive interfaces could be needed, potentially dwarfing the advantages of LCP.

According to a second aspect of the present invention, medical device is described to comprise a circuitry and a conductor arrangements according to an embodiment of the first aspect of the present invention. The circuitry includes a carrier substrate consisting of an LCP material, the carrier substrate carrying conductor lines and, optionally, electronic components interconnected by the conductor lines. The sheet substrate of the conductor arrangement is mechanically connected to the carrier substrate of the circuitry and the conductor lines of the conductor arrangement are electrically connected to the conductor lines of the circuitry.

According to a third aspect of the present invention, a method for fabricating an elongate conductor arrangement is described to comprise at least the following steps, preferably but not necessarily in the indicated order:
providing an elongate sheet substrate comprising a support layer consisting of a LCP material, arranging at least two conductor lines along a surface of the sheet substrate,
twisting the sheet substrate around a longitudinal axis of the sheet substrate into a spiral configuration while the sheet substrate being softened due to being subjected to an elevated temperature, and
reducing the temperature while the sheet substrate being in the spiral configuration.

Ideas underlying embodiments of the present invention may be interpreted as being based, inter alia, on the following observations and recognitions.

Briefly summarised in a non-limiting manner, embodiments of the present invention relate to the observation that an elongate conductor arrangement may be provided with high bending flexibility in various directions by specifically twisting an elongate LCP sheet substrate around its longitudinal axis to achieve a spiral configuration. Due to the twisted spiral configuration, the sheet substrate together with the conductor lines carried thereby may be easily bent in any direction, as there is generally always a small section in the elongate conductor arrangement at which the twisted sheet substrate extends orthogonal to a bending direction and may therefore be easily bent. As the entire conductor arrangement may be provided using LCP technology, it is compatible with an LCP-based circuitry in the medical device such that both components may be, for example, easily and reliably interconnected.

In the following, possible characteristics and advantages of embodiments of the present invention will be described in more detail.

Embodiments of the elongate conductor arrangement may be used for electrically connecting a first component to a second component, both components being arranged at positions spaced from each other. For example, a distance between both components may be between a few centimetres and many metres, sometimes even more. Particularly in medical applications, the distance may be typically between 1 cm and 10 m, preferably between 10 cm and 1 m. For example, the first component may be an electrode, a catheter, a sensor, an actuator, etc. The first component may be configured for being implanted into the patient's body. The second component may be a medical device. For example, the second component may be a pacemaker, an implantable cardioverter defibrillator (ICD), a neurostimulator, etc. The second component may be implanted at another position within the patient's body or may be provided external to the patient's body. Generally, the second component includes some circuitry. The circuitry may be configured for receiving, processing and/or sending electrical signals from and to the first component. Such signals may be transmitted along the conductor arrangement. At least portions of the conductor arrangement may be configured for being implanted into the patient's body.

An embodiment of the present invention may be an implantable pacemaker, an implantable cardioverter defibrillator (ICD) or an implantable neurostimulator connected to at least one electrode by the elongated conductor arrangement according to the first aspect of the present invention. The elongated conductor arrangement would function as electrode lead or lead of a pacemaker, ICD or neurostimulator in this embodiment of the present invention.

The sheet substrate of the elongate conductor arrangement has a quasi-two-dimensional geometry with its thickness being substantially smaller than its length and its width. For example, the width of the substrate is at least double its thickness, preferably at least five times its thickness. Furthermore, the sheet substrate has an elongate geometry with its length being substantially larger than its width. For example, the length of the substrate is at least 10 times its width, preferably at least 20 times or even at least 50 times its width.

The elongate sheet substrate comprises a support layer consisting of LCP material. Optionally, the entire elongate sheet substrates may consist of LCP material, i.e., the sheet substrate fully consisting of LCP material forms the support layer.

Liquid crystal polymers (LCPs) are polymers with the property of a liquid crystal, usually containing aromatic rings as mesogens. In other words, LCPs exhibit liquid crystalline properties in the melt (thermotropic) or in solution (lyotropic) and thus a certain degree of order. For a polymer to have liquid crystalline properties, mesogens must be present in the polymer. These can be located in the main chain as well as in side chains. The arrangement of mesogens in main chain LCPs leads to a rod-shaped molecular form. Such molecules are not very flexible. This results in extraordinary mechanical and chemical properties. Parallel to the molecular axis, LCPs have an extremely high tensile strength and a high modulus of elasticity, which predestines main-chain LCPs for use as high-performance fibres (e.g., protective clothing, sports equipment, space technology). In addition, the strongly anisotropic geometry ensures strong intermolecular cohesion, which means that melting points (if any) are correspondingly high and there is generally poor solubility. Therefore, precision components (e.g., scales, medical devices) can be moulded that, in addition to the mechanical properties mentioned above, retain their shape in the presence of water or organic solvents. Side-chain LCPs combine the properties of liquid crystals and those of polymers with flexible main chains. This arrangement leads to a fixation of the liquid crystalline properties of the mesogens, which are linked to the polymer main chain via, e.g., ester bonds.

Overall, LCP is a high performance material with excellent thermomechanical behaviour. Generally, it may be formed to any desired shape. At room temperature, thin LCP films and fibers may show mechanical properties close to steel. An operational temperature for LCP circuits may reach 190° C. Multiple, standard surface mounted technology (SMT) reflow and soldering operations are possible. Other benefits may be a low moisture absorption and chemical stability. LCP belongs to the polymer materials with the lowest permeability for gases and water. LCP may be bonded to itself, thereby allowing multilayer constructions with a homogenous structure.

In the elongate conductor arrangement presented herein, a plurality of conductor lines is arranged along at least one of the surfaces of the sheet substrate. Generally, the conductor lines may directly contact the LCP support layer. Alternatively, an intermediate layer may be interposed between the conductor lines and the LCP support layer. The conductor lines typically extend longitudinally along the length of the elongate sheet substrate. The conductor lines may be linear, may have a curved geometry or may have a combination of both including linear sections and curved sections. The conductor lines may extend in parallel to each other. The conductor lines may be made with any electrically conductive material. Preferably, the conductor lines are made with metal, preferably highly conductive metal such as copper, aluminium, silver, gold or mixtures or alloys thereof.

Standard PCB (Printed Circuit Board) equipment and processes may be used to process LCP films.

Multilayered substrates may be constructed with LCP films by laminating metallized and structured LCP cores with a lower melting point bond film. LCP substrates may be assembled with SMT components and sealed, e.g., with heat welded lids or frames from LCP to provide a homogenous, miniaturized and hermetic housing. A remarkable advantage of LCP technology is the possible combination of standard flexible substrate technology with the thermoplastic material properties. Generally, LCP may be the only thermoplastic material, which is fully compatible with PCB and thin film technology.

In a straightforward conventional approach (not according to the present invention) in which the LCP sheet substrate has a planar geometry, an elongate conductor arrangement using such planar sheet substrate would have very inhomogeneous bending characteristics. Particularly, bending forces acting onto the planar sheet substrate in a direction orthogonal to its extension plane would easily bend the sheet substrate, whereas bending forces acting in other directions, i.e., for example, forces acting in parallel to the extension plane, could hardly bend the sheet substrate but, instead, the sheet substrate would attempt to locally deform and/or twist in reaction to such forces.

Accordingly, substantive mechanical stress would be applied to the sheet substrate upon being submitted to such non-orthogonal bending forces. As a result, significant wear and/or material fatigue may occur in the planar sheet substrates, potentially leading to damages in the conductor arrangement and therefore negatively affecting a reliability of the conductor arrangement.

In an alternative conventional approach (not according to the present invention) same or similar to the approach disclosed in European Patent No. 3 181 188 B1, an elongate sheet substrate may be wound around a cylindrical core such as to obtain a coil geometry. Upon being provided with such coil geometry, the sheet substrate may be easily bent in any direction. However, bending actions may result in portions of the sheet substrate being tipped away from the coil structure. This may stress adjacent material such as adjacent isolation material. Such stress may result in increased wear and/or material fatigue. Furthermore, for forming the elongate sheet substrate with the coil geometry and forming a conductor arrangement of an intended length thereof, the sheet substrate has to be provided with a very substantive initial length. As the conductor lines provided on the sheet substrates typically have a small cross section, such substantive length may result in relatively high electrical resistances being established throughout the length of the entire conductor arrangement.

It may be seen as an important characteristics of the conductor arrangement presented herein that its elongate sheet substrate is not provided with a fully planar structure but at least comprises sections in which the sheet substrate is twisted around its longitudinal axis such as to obtain a spiral configuration. In such spiral configuration, the plane in which the sheet substrate extends rotates around the longitudinal axis of the elongate sheet structure when moving from substrate section to substrate section in the length direction of the sheet substrate. In other words, when moving along the elongate sheet substrate in its longitudinal direction, an orthogonal onto a substrate section successively rotates in a clockwise direction or in a counterclockwise direction.

With such twisted spiral configuration, when bending forces are applied to the elongate conductor arrangement, there is generally at least one substrate section, preferably a multiplicity of substrate sections, which extend in such a plane such that the bending forces are directed in parallel to the orthogonal onto such substrate section. Accordingly, the twisted sheet substrate may be easily bent in such oriented substrate sections. Particularly, the respective substrate sections may be bent without substantially laterally moving and/or tipping a remainder of the conductor arrangement in other substrate sections. Thus, mechanical stress acting onto the elongate sheet structure upon being bent may be minimised in directions not being orthogonal to an extension plane of each of its substrate sections. As a result, wear and/or material fatigue may be minimised upon repeatedly bending the conductor arrangement.

Furthermore, it may be important to note that the twisted helical structure and the conductor lines attached thereto are not applied to any arbitrary elongate sheet substrate but to an elongate sheet substrate comprising a support layer of LCP material. Materials used for other types of sheet substrates may be soft and/or stretchable. A sheet substrate of such soft or stretchable material may not sufficiently protect conductor lines attached thereto against excessive stretching and/or the formation. In contrast hereto, LCP material is very loadable and may hardly be stretched along an extension plane of an LCP sheet substrate. Accordingly, such LCP sheet substrate may be covered, lined or laminated with an electrically conductive material layer such as a metal layer. Optionally, portions of such metal layer may be removed subsequently in order to form conductor lines. Due to the low stretchability of the LCP material, such conductor lines are protected against being excessively stretched upon stretching forces being applied to the entire conductor arrangement.

According to an embodiment, the LCP material is a thermoplastic material. Alternatively or additionally, a layer of thermoplastic material is applied onto the support structure.

In other words, the elongate sheet substrate itself or the support layer thereof may be provided with an LCP material having thermoplastic characteristics. Alternatively or additionally, a cover layer may be applied on top of the LCP support structure, such layer having thermoplastic characteristics. Having such thermoplastic characteristics, the sheet substrate, its support layer and/or the additional cover layer may be temporarily softened or even liquefied upon being submitted to elevated temperatures. Thereby, the substrate or layer may be attached or welded to other portions of the same substrate or layer or to portions of, for example, a circuitry made with LCP technology having an LCP material substrate. Additionally or alternatively, the elongate sheet substrate may be, for example, laminated in a hermetically tight manner to another thermoplastic additional cover layer, thereby enclosing and protecting the conductor lines included between the sheet substrate and the additional cover layer. Furthermore, due to the thermoplastic characteristics, the elongate sheet substrate may be easily twisted into its spiral configuration at elevated temperatures and the spiral configuration may then be solidified upon cooling down the thermoplastic material.

According to an embodiment, the sheet substrate is twisted with at least ten, preferably at least twenty or at least fifty, 360°-rotations per meter of length of the sheet substrate.

Expressed differently, in the elongate conductor arrangement described herein, the elongate sheet substrate may comprise a spiral configuration in which its extension plane is twisted in a 360°-rotation along a section of the sheet substrate having a length of 10 cm or less, preferably 5 cm or less or even 2 cm or less. Accordingly, as will be described with reference to specific calculations further below, upon being submitted to bending forces, the elongate conductor arrangement may easily and quasi-continuously be bent even into small bending radii of, for example, less than 10 cm or even in a range of between 1 cm and 5 cm, as there is at least one or preferably multiple partial sections in the elongate conductor arrangement at which the elongate sheet substrate extends within an extension plane being orthogonal to the bending forces and may therefore be easily bent.

According to an embodiment, the conductor arrangement further comprises a cover layer extending along the surface of the sheet substrate and covering the conductor lines.

Such cover layer may comprise or consist of a polymer, preferably an LCP. For example, the cover layer may consist of the same LCP material as the support layer of the sheet substrate. The cover layer may cover at least portions or, preferably, an entire area of one of the main surfaces of the sheet substrate. The cover layer may be attached to the sheet substrates, for example, with a positive junction jointing, using, e.g., welding techniques. The cover layer together with the sheet substrate may enclose the conductor lines comprised in between in a tight manner, preferably in a fluid tight manner. Accordingly, the conductor lines may be protected against mechanical and/or chemical attacks.

According to an embodiment, at least one of the conductor lines is arranged along each of opposing main surfaces of the sheet substrate.

In other words, the sheet substrate may carry conductor lines not only along one of its main surfaces but along both opposing main surfaces. In such configuration, a distribution of forces or mechanical tensions acting onto the sheet substrate and the conductor lines may be more symmetrical upon, for example, bending the entire conductor arrangement in opposing directions, as compared to a case where conductor lines are applied to only one of the main surfaces of the sheet substrate. A bending axis may therefore be located approximately in the middle of the sheet substrate. As a result, a packing density of conductor lines may be increased.

According to an embodiment, the conductor lines are arranged along the surface of the sheet substrate in a configuration being symmetrical with regards to a middle line extending longitudinally along a center of the sheet substrate, and/or with regards to a middle plane extending in parallel to opposing main surfaces of the sheet substrate.

In the first option, a same number of conductor lines is arranged at same distances and same geometrical configurations at both laterally opposing sides of the middle line of the sheet substrate. In the second option, a same number of conductor lines is arranged at same distances and same geometrical configurations at both opposing main surfaces of the sheet substrate with regards to the central middle plane of the sheet substrate. In both cases, forces or mechanical tensions acting onto the conductor arrangement, for example, upon bending same may be distributed symmetrically. Thereby, wear and/or material fatigue in the conductor arrangement may be reduced and/or a packing density of conductor lines may be increased.

According to an embodiment, the conductor arrangement includes a layer stack comprising at least two elongate sheet substrates stacked on top of each other, each sheet substrate carrying at least one conductor line arranged along a surface of the respective sheet substrate.

In such implementation, the conductor arrangement includes at least two sets of conductor lines, each set extending in one of parallel planes and being separated from a neighboring set by an interposed one of the sheet substrates. Having such plural sets of conductor lines, a total number of conductor lines in the conductor arrangement may be significantly increased. In the layer stack, the various sheet substrates each carrying its conductor lines may be attached to each other such as to, e.g., form an integrated unit. For example, neighboring sheet substrates may be mechanically fixed to each other via intermediate thermoplastic sheets acting as a gluing layer. The entire layer stack may then be twisted around a longitudinal axis of at least one of the sheet substrates into the spiral configuration.

According to a further specified embodiment, each of the sheet substrates in the layer stack carries a same number of plural conductor lines.

In other words, the layer stack may be formed using multiple sheet substrates of a same type, i.e., each sheet substrate carrying the same number of conductor lines and, preferably, having a same geometry. A cross-section of such layer stack may be rectangular. By stacking such sheet substrates on top of each other, the total number of conductor lines in the conductor arrangement may be multiplied in a simple manner.

According to a further specified embodiment, a central one of the sheet substrates in the layer stack carries a larger number of conductor lines than a peripheral one of the sheet substrates. Additionally or alternatively, a central one of the sheet substrates has a larger width than a peripheral one of the sheet substrates.

In the first option, in the layer stack forming the elongate conductor arrangement, one or more peripheral sheet substrates are carrying smaller numbers of conductor lines than the central one of the sheet substrates. This may result in more homogeneous bending characteristics of the conductor arrangement, i.e., the conductor arrangement may be bent in various directions, wherein forces acting onto the sheet substrates included in the layer stack are distributed more homogeneously and peak forces may be avoided in a more effective manner than compared to a configuration in which the peripheral sheet substrates comprise a same number of conductor lines as the central sheet substrate.

In the second option, a similar effect as described for the first option may be obtained by providing the peripheral sheet substrates with a smaller width than the central sheet substrate. Having such smaller width at its periphery, the entire conductor arrangement may obtain a non-rectangular cross-section such as a quasi-elliptical or quasi-circular cross section.

According to an embodiment, the conductor arrangement further comprises a matrix material enclosing the one or more sheet substrates and the conductor lines such that the conductor arrangement has a circular cross section.

In other words, while each of the sheet substrates generally has a rectangular cross-section, the single sheet substrate or a stack of several sheet substrates may be enclosed in additional matrix material such as to obtain a circular cross section. The matrix material may be, for example, a polymer, preferably a thermoplastic polymer. For example, the matrix material may be applied to the sheet substrates using extrusion or immersion techniques. Having a circular cross-section, the entire conductor arrangements may be improved with regards to its bending characteristics and/or with regards to its sliding characteristics upon, for example, been included in a lumen of a tube.

According to an embodiment, the conductor arrangement further comprises a tube enclosing the substrates and the conductor lines.

For example, the twisted conductor arrangement may be included into the lumen of a tube or may be extruded into the tube during an extrusion procedure. The tube may therefore protect the enclosed conductor arrangement. Optionally, the conductor arrangement may be held within the tube such as to enable axial motion between both components.

According to an embodiment, the twisted sheet substrate is provided with a coiled geometry.

Expressed differently, the sheet substrate itself may be twisted into the spiral configuration and then this twisted sheet substrate may be wound around a cylindrical core into a coil configuration. Accordingly, a multipolar rotated sheet conductor arrangement is generated having a shape of a coil. Therein, forces acting onto the included conductor lines may be further reduced as compared to a non-coiled configuration, as bending forces onto a conductor line in a coil shape generally only induce torsional loads onto the conductor line. As the conductor arrangement itself is already twisted, it is well suited to absorb such torsional loads.

According to an embodiment, the conductor arrangement further comprises an electrode, wherein the electrode is formed by a protrusion integrally protruding from the sheet substrate and carrying an electrode layer electrically connected to one of the conductor lines.

In other words, the elongate conductor arrangement may comprise protrusions which are integrally formed by the sheet substrate and at which the sheet substrate further extends beyond its general lateral edges. Such protrusion carries an electrode layer made from an electrically conductive material such as, for example, the material which is also forming the conductor lines. The electrode layer is electrically connected to one of the conductor lines such that an electrical potential applied to the respective conductor line is induced at the electrode layer. Accordingly, the electrode forming an integral part of the proposed conductor arrangement may be used for applying electrical voltages, for example, at a location within a patient's body at which the conductor arrangement is located with its electrode. For such purpose, the electrode layer may be exposed, i.e., any cover layer covering the sheet substrate of the conductor arrangement may be locally removed for exposing the electrode layer.

In embodiments of the medical device according to the second aspect of the present invention, a circuitry includes a carrier substrate consisting of an LCP material, the carrier substrate carrying electronic components and conductor lines interconnecting the electronic components. The sheet substrate of the conductor arrangement is mechanically connected to the carrier substrate of the circuitry and the conductor lines of the conductor arrangement are electrically connected to the conductor lines of the circuitry. For example, as the carrier substrate of the circuitry and the sheet substrate of the conductor arrangement may preferably consist of a thermoplastic LCP material, both components may be mechanically interconnected, for example, by welding, thereby forming a reliable positive junction jointing between both components. Therein, various advantages of the LCP technology may be beneficially used.

In embodiments of the method for fabricating an elongate conductor arrangement according to the third aspect of the present invention, conductor lines are arranged along a surface of a sheet substrate. For example, the conductor lines may be deposited using various techniques which are generally applicable in LCP technology, including inter alia deposition techniques such as printing techniques (e.g., screen printing, roller printing, inkjet printing, etc.), CVD techniques (chemical vapor deposition), PVD techniques (physical vapor deposition), photolithography, etc. The sheet substrate is then heated to elevated temperatures of, for example, more than 80° C., preferably more than 100° C., more than 130° C. or even more than 160° C. in order to temporarily soften the LCP material being preferably thermoplastic. In such softened condition, the sheet substrate is then twisted around the longitudinal axis of the sheet substrate into the spiral configuration. Subsequently, the temperature is reduced again while the sheet substrate being in the spiral configuration, thereby solidifying the spiral configuration.

According to a specific implementation, the softened elongate sheet substrates may be guided through one or more slit diaphragms and/or a pair of parallel axles being arranged in a twisted configuration, i.e., rotated with respect to each other. Simultaneously or subsequently, the sheet substrate twisted thereby is cooled down and is thereby solidified in its spiral configuration.

According to an alternative implementation, the softened elongate sheet substrate may be held in a long device and may be controllably twisted by rotating its ends relative to each other end before subsequently cooling down and solidifying the twisted configuration.

It shall be noted that possible features and advantages of embodiments of the present invention are described herein with respect to various embodiments of the elongate conductor arrangement, on the one hand, and embodiments of the medical device comprising such conductor arrangement or embodiments of a method for fabricating such conductor arrangement, on the other hand. One skilled in the art will recognize that the features may be suitably transferred from one embodiment to another and features may be modified, adapted, combined and/or replaced, etc. in order to come to further embodiments of the present invention.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

In the following, advantageous embodiments of the present invention will be described with reference to the enclosed drawings. However, neither the drawings nor the description shall be interpreted as limiting the present invention.

FIG. 1A, 1B show a cross-sectional view and a top view, respectively, of an elongate conductor arrangement.

FIG. 2 shows an elongate conductor arrangement in a spiral configuration according to an embodiment of the present invention.

FIG. 3 shows a perspective view of a portion of an elongate conductor arrangement including a cover layer.

FIG. 4 visualises in cross-sectional views a fabrication of a conductor arrangement comprising a stack of several sheet substrates.

FIG. 5 shows an elongate conductor arrangement comprising a stack of several sheet substrates in a spiral configuration according to an embodiment of the present invention.

FIG. 6 shows a perspective view of the elongate conductor arrangement of FIG. 5 with conductor lines being locally exposed.

FIG. 7 shows a cross-sectional view of a conductor arrangement having a circular cross section.

FIG. 8 shows a portion of a medical device including a conductor arrangement according to an embodiment of the present invention.

FIG. 9 shows a portion of a medical device including a conductor arrangement according to another embodiment of the present invention.

FIG. 10 shows a conductor arrangement according to another embodiment of the present invention included in a tube.

The figures are only schematic and not to scale. Same reference signs refer to same or similar features.

### DETAILED DESCRIPTION

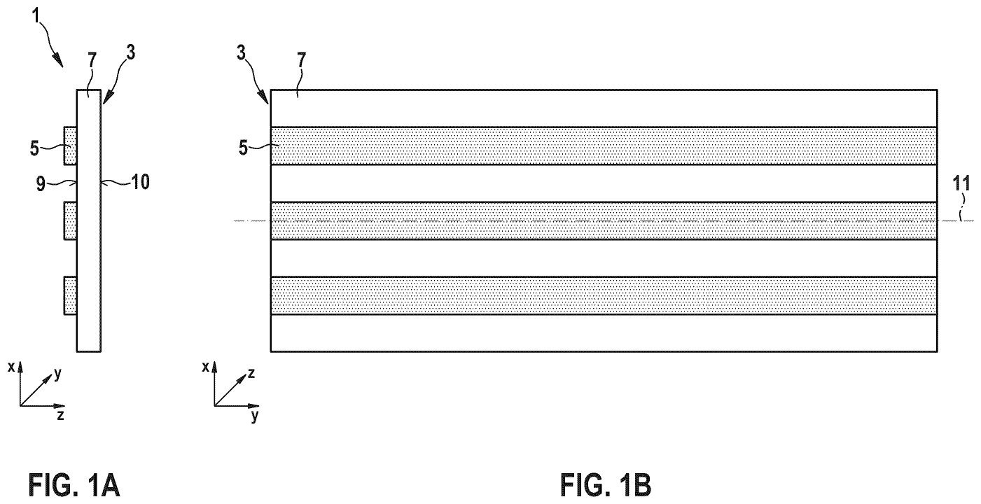

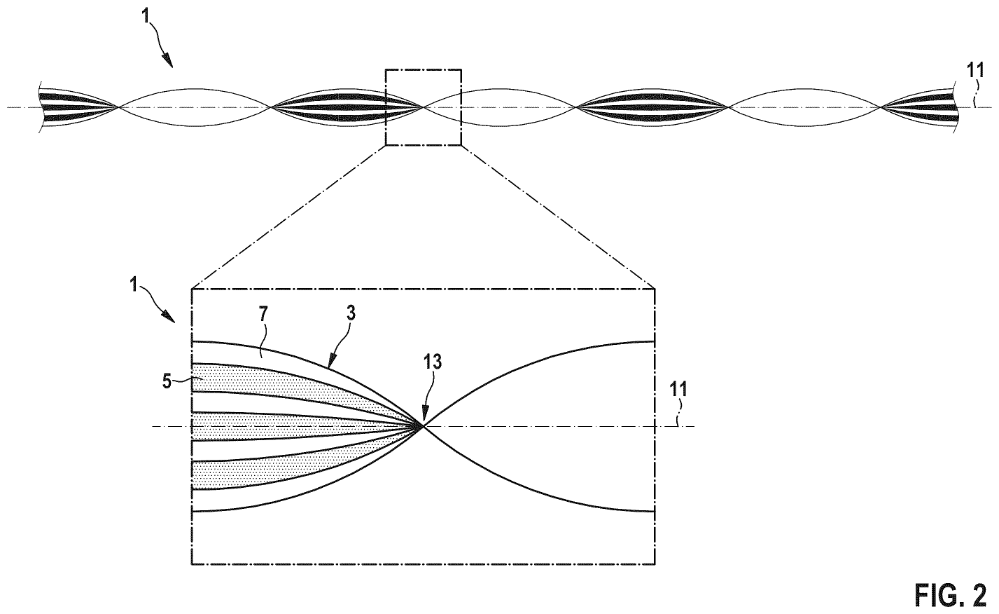

FIGS. 1A, 1B and FIG. 2 show an elongate conductor arrangement 1 which may be used for establishing an electrical connection to a medical device. The conductor arrangement 1 is referred to herein sometimes as conductor track. The conductor arrangement 1 comprises an elongate sheet substrate 3 and the several conductor lines 5. The sheet substrate comprises or consists of a support layer 7 consisting of LCP material. The conductor lines 5 are arranged along a surface 9 of the sheet substrate 3. As visualised in FIG. 2, the sheet substrate 3 is twisted around a longitudinal axis 11 of the sheet substrate 3 such as to obtain a spiral configuration.

First, some basic principles underlying the conductor arrangement described herein will be summarized as follows:

The elongate conductor arrangement proposed herein is based on the idea to make LCP (liquid crystal polymer) conductive tracks usable as elongated, flexible leads.

Conductive tracks formed by conductor lines 5 on a flexible LCP sheet substrate 3 forming a carrier are very attractive for use, e.g., in electrodes and catheters because implantable products with a high conductor density and the ability to integrate a wide range of circuit options can be constructed using simple technologies that can be easily automated.

The metallic conductor lines 5 embedded in the carrier material usually have a very small material thickness (nanometres to a few micrometres) and are correspondingly sensitive to buckling stress. In order to prevent sharp kinking of the conductor lines 5, additional insulation materials are usually necessary as kink protection (e.g., in the form of tubes made of silicone Si, polyurethane PU, etc.).

However, LCP sheet tracks have a strong (long axis) and a weak bending axis (short axis) due to their flat orientation when bent. This asymmetry means that when bending at an angle to the weak axis, the track tends to twist, so that the bending always takes place via the weak axis. Bending changes thus lead to changes in the position of the conductor lines 5 and, in the case of adjacent insulation materials, to an increased risk of abrasion.

As a rule, in conventional approaches for electrically connecting a medical device, it is therefore necessary to use other types of lead-in concepts such as wires, wire coils or ropes. The need to combine several technologies generally severely limits the possibilities of this promising new LCP-based material.

The solution according to embodiments of the present invention is to design the flat conductor in such a way that the asymmetric bending behaviour is homogenised and torsions are suppressed. One possible solution is to anticipate the torsion of the conductor in the manufacturing process and to torsion the LCP in itself into a spiral configuration like a DNA. In this state, it has at least one corresponding weak bending plane in each possible bending direction, which can absorb the bending stresses without the conductor arrangement having to change its position.

Subsequently, embodiments of the conductor arrangement 1 and of a medical device provided therewith as well as of a fabrication method will be described in more detail as follows:

LCP conductor tracks are very attractive for application in electrodes and catheters because implantable products with a high conductor density and an integrability of diverse circuit options may be constructed with simple, automatable technologies. However, LCP tracks may generally only be bent in one plane due to their flat orientation and are therefore only very limitedly suitable for the design of long flexible leads. Therefore, it is usually necessary to use a different type of lead-in concept such as wire, wire helix or cable. The need to combine several technologies severely limits the possibilities of this promising new material and the use of its economic potential.

The task is to design LCP conductors in such a way that they are flexible and bendable independent of the plane. In this way, it is possible to do without connecting different conductor concepts with each other.

Conventionally, the aforementioned supply lines such as wire, helix or rope must generally be connected to the LCP components in order to make the LCP technology available. This requires connection technologies that make both the production and the product very bulky, expensive and risky.

If the conductive track is designed as a helix, it is permanently stable, but when bent, it causes the conductive track to tilt out of the helix structure, which in the longer term causes adjacent insulation materials to fail. The filament of a spiral transforms a compressive, tensile or bending stress into a torsion of the filament. A round wire as filament is very stable under this type of stress because it is symmetrically elastically torsionable. However, if the filament is a flat conductor, it tilts out of the axial plane of the helical structure. This movement must be provided for in the design of the conductor so that unacceptable stress does not result from the load on the insulation material and the flat conductor. In addition, a large initial length of the track is required for coiling, which, with the conductor cross-sections, which are, as expected, significantly smaller than those of classic wire coils, causes the resistance of the supply line to rise sharply.

The aim of the conductor arrangement described herein is to offer a multipolar, highly flexible lead-in that exclusively uses LCP track technology and can thus be ideally combined and connected with other LCP components. The production can be carried out with uniform very efficient processes. The products benefit from the miniaturisation possibilities and the high packing density of LCP technology.

FIG. 1 describes the initial situation. The elongate sheet substrate 3 of preferably thermoplastic carrier material, e.g., made of an LCP, has several longitudinally arranged conductor lines 5. FIG. 1A shows the arrangement in cross-section. It is drawn in the x-z plane. FIG. 1B is a top view of the conductor arrangement in the x-y plane. Stripe-like conductor lines 5 are seen on the sheet substrate 3.

The disadvantage of this design is the limited bendability in space. The conductor arrangement 1 is very flexible in the z-direction, but in the y-direction the conductor arrangement 1 avoids bending due to torsion.

FIG. 2 describes the modification according to an embodiment of the present invention of the flat conductor arrangement 1 shown in FIG. 1. The conductor arrangement 1 is rotated around its longitudinal axis 11, i.e., around its central axis. The outer edges of this three-pole flat conductor then form a double helix and are reminiscent of the rough structure of a DNA. The plane of the flat conductor arrangement 1 was spanned only in the x-y plane in FIG. 1, i.e., before the modification. Now this plane rotates along the centre axis in the y-z plane. If the axis of the flat conductor arrangement 1 is now bent in a plane, conductor sections are found for any bending direction that are particularly easy to bend because they are orthogonal to the bending direction at this point. Since the conductor can be bent easily in these zones, the bending stress is relieved here and cannot form folds or stress damage in those zones that cannot be bent for the present bending direction. If FIG. 2 is shown in the x-y plane, then nodes 13 shown are where bending would occur if bending were to occur in the x-y plane.

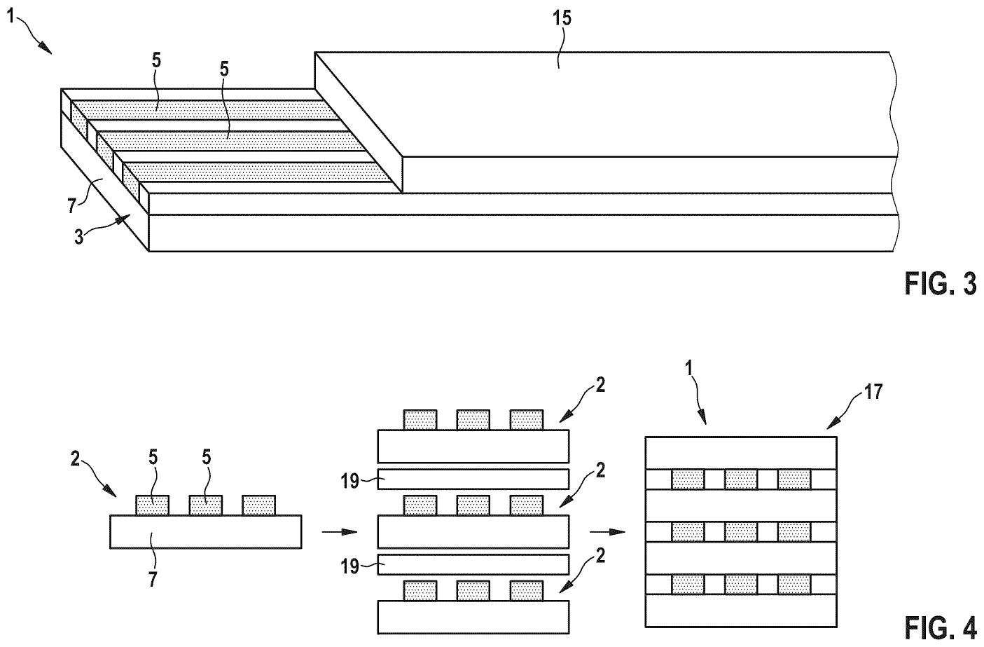

It makes sense that the conductor lines 5 are additionally insulated by a cover layer 15 as shown in FIG. 3. The material thickness, which can be seen in FIG. 3, is neglected in the sketches in FIG. 2 for a clear representation.

Considerations regarding the number of rotations around the longitudinal axis 11:

The smallest radius to which the multipolar, initially flat conductor arrangement 1 is to be subjected is, for example, R=1 cm. If, for example, eight bending points are to be provided in the conductor for a 360° bend with this radius, so that the conductor arrangement follows the bend smoothly, the conductor arrangement must make four rotations over the length of the bend (2*Pi*1 cm), because during a full rotation the conductor plane is perpendicular to the bending axis two times. The conductor arrangement must therefore complete a 360° rotation around its own axis within 1.57 cm (which corresponds to a quarter of the length of the bend) for the above condition. With a typical electrode length of 60 cm, the number of rotations is then about 38.

When the conductor arrangement 1 rotates around its own axis, the edges of the conductor arrangement 1 are slightly stretched because they now take a helical course. The stretching towards the edges depends on the number of rotations and the width of the conductor arrangement 1. If, in the presented case, the width of the conductor arrangement 1 is assumed to be 1 mm, then the edges of the conductor arrangement 1 experience a strain of approx. 2%. Conductor lines 5 that lie away from the centre axis of the conductor arrangement 1 would experience a reduction in cross-section, which can be compensated for by increasing the line dimensions, e.g., its width.

In one embodiment, the elongate sheet substrate 3 is provided with conductor lines 5 on both sides, i.e., at both opposing main surfaces 9, 10. The conductor lines 5 may be directly opposite each other or staggered. In this variant, the stress distribution of the flat conductor arrangement 1 is symmetrical in both bending directions. The bending axis remains in the centre of the insulating sheet substrate 3 when bending in both directions, unlike with an asymmetrically coated carrier sheet substrate 3. In this version, the packing density of the feed conductor lines 5 may be increased.

FIG. 4 shows a variant with which the packing density of the conductor lines 5 may be significantly increased by layering several flat partial conductor arrangements 2, thereby forming a layer stack 17 generating an overall conductor arrangement 1. The individual flat partial conductor arrangements 2 with their insulating sheet substrates 3 and their conductor lines 5 are positioned on top of each other and welded together, e.g., by means of a thermoplastic mouldable foil 19, whose melting point is below that of the LCP material of the sheet substrate 3. In the example shown in FIG. 4, three flat partial conductor arrangements 2 with three conductor lines 5 each are layered. They then form a 9-pole overall conductor arrangement 1 with a square cross-section.

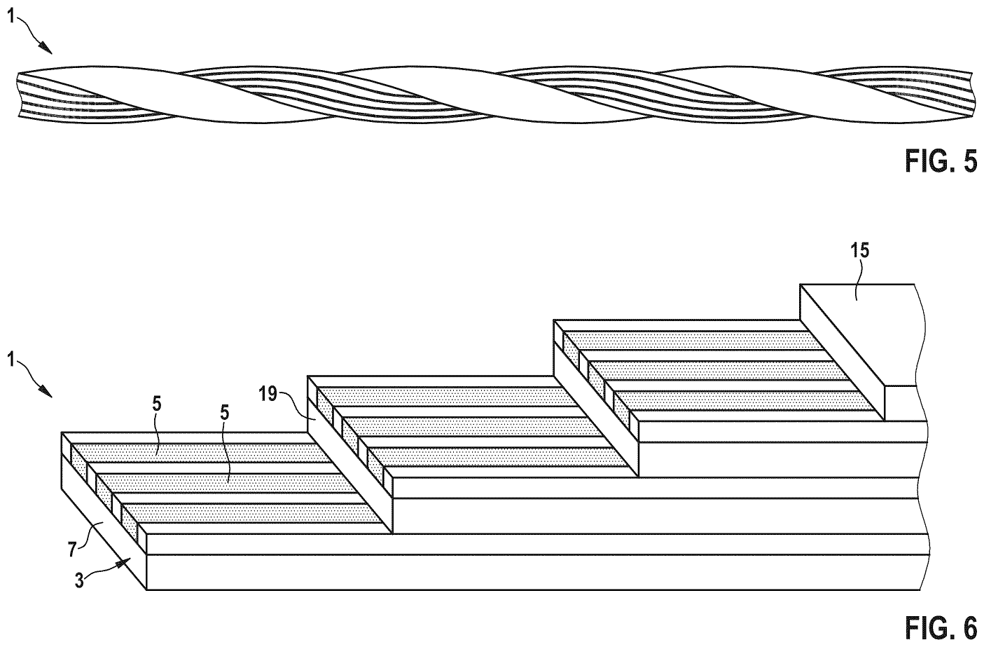

The overall conductor arrangement 1 may then be twisted into a spiral configuration as depicted in FIG. 5.

In order to make the conductor lines 5 accessible for contacting, at least some of the layers formed by the sheet substrates 3 and/or by the mouldable foils 19 may be locally removed, thereby locally exposing the conductor lines 5. For contacting, e.g., further flat conductor arrangements may be thermoplastically welded to the insulating sheet substrate 3 of the conductor arrangement 1 and connected to the conductor lines 5, e.g., by means of through-hole plating. FIG. 6 shows the layers exposed in stages.

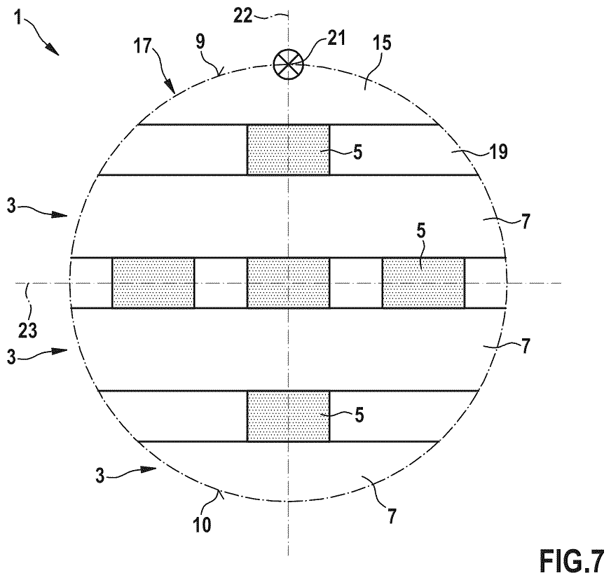

As shown in FIG. 7, in a further design with several layered partial conductor arrangements 2, the number of conductor lines 5 in the different levels varies. The above example from FIG. 4 may be varied in such a way that the lower level has, e.g., only one conductor line 5, the middle level has three conductor lines 5 and the upper level again only one conductor line 5, as shown in FIG. 7. Particularly, similar to the layer stack 17 in FIG. 4, the conductor lines 7 may be arranged in symmetrical configurations with regards to a center plane 22 including a middle line 21 extending longitudinally along a center of the sheet substrate 3 and/or with regards to a middle plane 23 extending in parallel to opposing main surfaces 9, 10 of the sheet substrate 3. Since insulation material may then also be reduced at the edges of the cross-section, such a construct can be converted into a round conductor cross-section, for example.

In another version, the conductor arrangement I may be rolled along its longitudinal axis 11 and joined together at the edges. For the necessary bend protection, a flexible insulation material, e.g., silicone or PU, may be placed in the resulting lumen of the conductor tube. The diameter of this construction should not exceed 1 mm, preferably 0.5 mm.

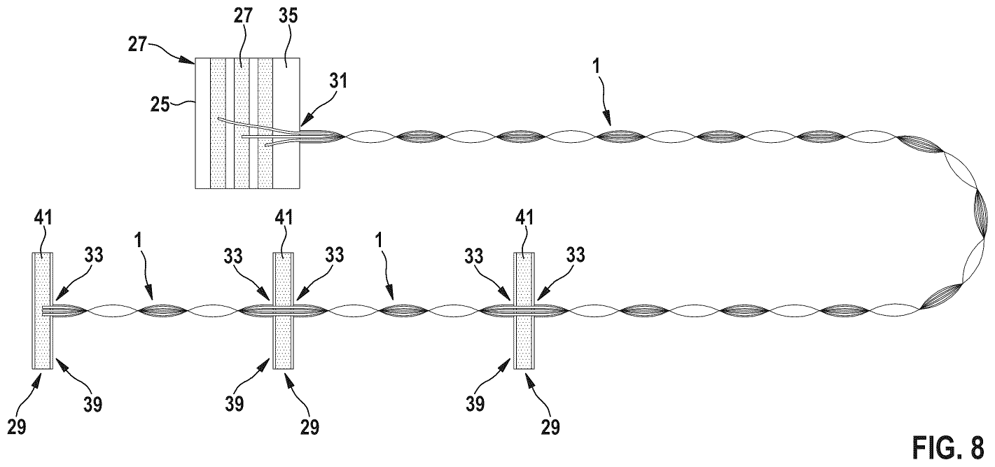

FIG. 8 shows an example of an implementation in a fictitious product in which a portion of a circuitry 25 of a medical device 27 is electrically connected to, for example, electrodes 29 by an elongate conductor arrangement 1. The purpose is to present a possible assembly concept, not a product. FIG. 8 shows components for the construction of a 3-pole electrode assembly with three electrodes 29.

One component is the described twisted conductor arrangement 1, which may be produced, e.g., continuously by the metre. The twisting of the strip can also be done in a continuous process.

In order to build up the product from it, the twisted conductor arrangement 1 is cut into required lead sections. Proximal ends 31 and/or distal ends 33 of these sections are exposed, e.g., with a laser. The circuitry 25 forming a separate LCP module is provided for a connector with a carrier substrate 35 carrying conductor lines 37. The conductor lines 37 of the circuitry 25 on one side may connect to the conductor lines 5 of the twisted conductor arrangement 1. It is welded and contacted to the exposed part of the twisted conductor arrangement 1. Conductive lines are applied to the rear side of the connector circuitry 25, which may represent contact zones of the connector. These contact zones may be connected to surrounding structures, e.g., by means of through-plating. Each conductive line leads to a contact strip. In a later process, the connector circuitry 25 may be rolled into a sleeve with conductive rings on the outside.

For the implementation of the ring electrodes 29, three identical protrusions 39 may be provided, which are also provided with a conductive electrode layer 41 on both sides. The protrusions 39 are welded in the transition ends 33 on each side to a twisted conductive conductor arrangement 1. In the case of the terminating electrode 29, it is left open how the electrode 29 is further routed. As a special feature, the electrodes 29 formed by the protrusion 39 may be passed through and transferred to the next twisted conductor arrangement 1. The position of the through-plating on the respective electrode 29 decides to which conductor arrangement 1 the electrode surface is connected.

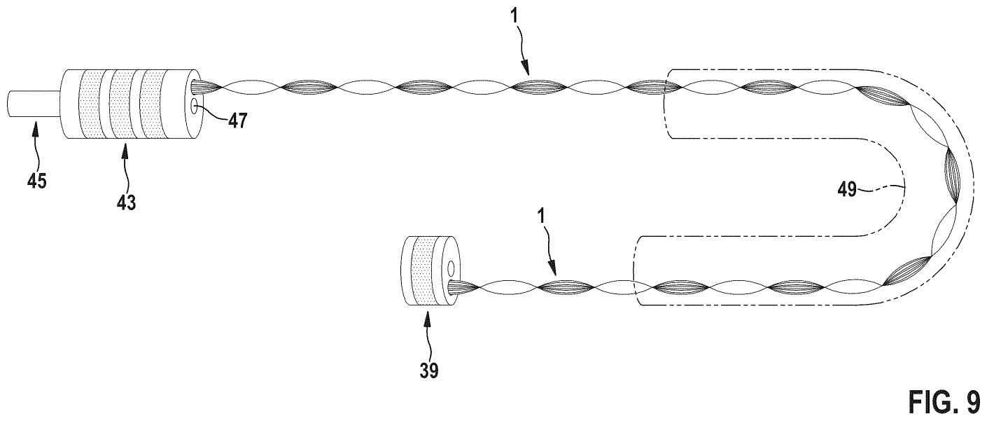

FIG. 9 describes how the components from FIG. 8 may be represented in a product.

The connector 43, thermoplastically rolled from the flat connector circuitry 25 (FIG. 8) and formed into a sleeve, has been given a connector pin 45 as an example and is thus reminiscent of an IS4 connector. The concept shown here could also use a centrally guided inner helix and thus make the electrode a 4-polar product. However, an inner lumen 47 may also remain free and only be used as a central guiding lumen for a stylet, a guidewire or for the administration of contrast medium. Only the course of the first twisted conductor arrangement 1 is shown. In the centre of the electrode, an insulation tube 49 is only schematically indicated, which is designed as a multi-lumen tube. In this illustration, the electrode ends with a ring electrode 29 thermoplastically formed from the flat LCP protrusions 39 (FIG. 8). The further distal structure of the electrode is not shown here.

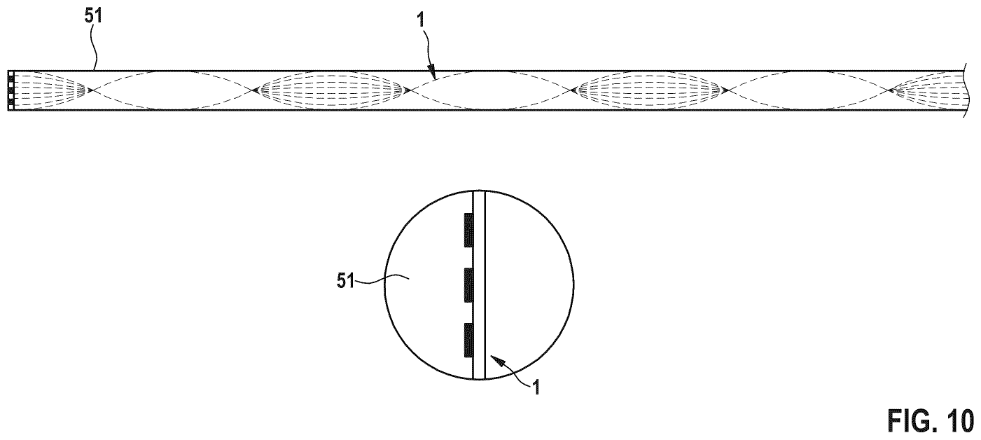

FIG. 10 shows, in a longitudinal sectional view and in a cross sectional view, an implementation variant in which the twisted flat conductor arrangement 1 is over-extruded with LCP matrix material 51. Here, the finished twisted conductor arrangement 1 is fed and embedded during the extrusion of an LCP strand. This design protects the twisted flat conductor arrangement 1. It may be guided in a lumen where it may move axially if necessary without exposing the edges of the flat conductor to friction. This implementation variant may also transmit torsional forces. A flat conductor arrangement 1 embedded in a round body in this way may also be additionally coiled.

FIGS. 8 and 9 show a proposal for the application of embodiments according to the present invention in a product. Many other implementation possibilities are conceivable. Further implementations may, e.g., include:
The twisted conductor arrangement 1 is guided centrally in the electrode. This is useful if the central lumen is not needed for a stylet or a rotatable inner helix.
Several twisted conductor arrangements 1 are used in parallel.
The flat conductor arrangement 1 is coated by dipping, over-extrusion or tubing. In one design, the twisted flat conductor arrangement 1 is filled to form a round body. This may provide better sliding properties in a hose lumen.
The twisted flat conductor arrangement 1 is embedded in the lumen of a tube or extruded into a tube during extrusion.
In one embodiment, the multipolar flat conductor arrangement 1, which is twisted as above, is additionally coiled. The result is a multipolar rotated flat conductor that forms the shape of a helix. This construction leads to a further relief of the conductor structure, because bending stresses of a coiled conductor are only converted into a torsional stress of the same conductor. Since the conductor arrangement 1 itself is already torsionally stressed from the start, it is predestined for this stress.
Sensors, actuators, energy-storing or energy-generating elements, switching, amplifier, communication, data-storing or data-processing elements may be integrated into the conductor concept.
The arrangement of the conductor lines may have structures that suppress/attenuate a coupling of MRI energies.
One variant is a flat conductor arrangement 1 with sections that are twisted to different degrees. This design may be used if it is known where special bends are to be expected on the electrode. For example, tight bending radii can quickly occur in the connector area if the electrode is bent over a header edge. Here, the flat conductor arrangement 1 is particularly twisted so that there are enough bending points to absorb the bending stresses at the imposed points and bend the conductor arrangement 1 without stress.
The sheet substrate and/or an embedding may be made of one or a combination of following materials: Polyurethane (PU), Polyester Urethanes (PEU), Polyether Urethanes (PEEU), Polycarbonate Urethanes (PCU), Silicone based Polycarbonate Urethanes (PCU) bonds better with silicone both extrusion, polycarbonate polyurea urethanes (PCHU), polydimethylsiloxane urethanes (PSU), polyisobutylene urethanes (PIU), polyisobutylene-based copolymers (PIC), polyether block amides (PEBA, for example, PEBAX), polyimides (PI), fluorinated hydrocarbons, ethylene-tetrafluoroethylene copolymer (ETFE), polytetrafluoroethylene (PTFE), polysulfone (PSU), polyethylene (PE), polypropylene (PP), polyamides (PA), silicone, polyimides (PI), fluorinated hydrocarbons, ethylene-tetrafluoroethylene copolymer (ETFE), polytetrafluoroethylene (PTFE), tetrafluoroethylene (TFE), perfluoro (ethylene-propylene) (FEP), perfluoroalkoxy polymers (PFA), PEEK.

Embodiments of the proposed conductor arrangement 1 may be fabricated, e.g., as follows:
The flat conductor arrangement 1 may be produced by lamination of several partial conductor arrangements 2. In a softened state at elevated temperatures, a flat strip may be guided through one or more slotted apertures twisted to the direction of lamination or correspondingly arranged closely parallel shafts and may then be cooled in the process for solidifying the resulting helical configuration.
The flat conductor arrangement 1 may be clamped in a long device, twisted in a controlled manner and annealed in the twisted state.

Finally, it should be noted that the term “comprising” does not exclude other elements or steps and the “a” or “an” does not exclude a plurality. Also elements described in association with different embodiments may be combined. It should also be noted that reference signs in the claims should not be construed as limiting the scope of the claims.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

### LIST OF REFERENCE NUMERALS

1 conductor arrangement
2 partial conductor arrangement
3 elongate sheet substrate
5 conductor line
7 support layer
9 main surface
10 opposing main surface
11 longitudinal axis
13 node
15 cover layer
17 layer stack
19 mouldable foil
21 middle line
22 center plane
23 middle plane
25 circuitry
27 medical device
29 electrode
31 proximal end of lead section
33 distal end of lead section
35 carrier substrate
37 conductor lines
39 protrusion
4 electrode layer
43 connector
45 connector pin
47 inner lumen
49 insulation tube
51 matrix material

1. An elongate conductor arrangement for electrical connection to a medical device, comprising:
an elongate sheet substrate,
at least two conductor lines,
wherein the elongate sheet substrate comprises a support layer consisting of a LCP material,
wherein the conductor lines are arranged along a surface of the sheet substrate,
wherein the sheet substrate is twisted around a longitudinal axis of the sheet substrate into a spiral configuration.

2. The conductor arrangement according to claim 1, wherein at least one of the following conditions applies:
the LCP material is a thermoplastic material,
a layer of thermoplastic material is applied onto the support structure.

3. The conductor arrangement according to claim 1, wherein the sheet substrate is twisted with at least ten 360°-rotations per meter of length of the sheet substrate.

4. The conductor arrangement according to claim 1, further comprising a cover layer extending along the surface of the sheet substrate and covering the conductor lines.

5. The conductor arrangement according to claim 1, wherein at least one of the conductor lines is arranged along each of opposing main surfaces of the sheet substrate.

6. The conductor arrangement according to claim 1, wherein the conductor lines are arranged along the surface of the sheet substrate in a configuration being symmetrical with regards to at least one of
a middle line extending longitudinally along a center of the sheet substrate (3), and
a middle plane extending in parallel to opposing main surfaces (9, 10) of the sheet substrate.

7. The conductor arrangement according to claim 1, wherein the conductor arrangement includes a layer stack comprising at least two elongate sheet substrates stacked on top of each other, each sheet substrate carrying at least one conductor line arranged along a surface of the respective sheet substrate.

8. The conductor arrangement according to claim 7, wherein each of the sheet substrates in the layer stack carries a same number of plural conductor lines;

9. The conductor arrangement according to claim 7, wherein at least one of the following conditions applies:
a central one of the sheet substrates in the layer stack carries a larger number of conductor lines than a peripheral one of the sheet substrates;
a central one of the sheet substrates has a larger width than a peripheral one of the sheet substrates.

10. The conductor arrangement according to claim 1, wherein the conductor arrangement further comprises a matrix material enclosing the sheet substrate and the conductor lines such that the conductor arrangement has a circular cross section.

11. The conductor arrangement according to claim 1, wherein the conductor arrangement further comprises a tube enclosing the sheet substrate and the conductor lines.

12. The conductor arrangement according to claim 1, wherein the twisted sheet substrate is provided with a coiled geometry.

13. The conductor arrangement according to claim 1, further comprising an electrode, wherein the electrode is formed by a protrusion laterally protruding from the sheet substrate and carrying an electrode layer electrically connected to one of the conductor lines.

14. A medical device comprising a circuitry, and a conductor arrangement according to claim 1,
wherein the circuitry includes a carrier substrate consisting of an LCP material, the carrier substrate carrying conductor lines and, optionally, electronic components interconnected by the conductor lines,
wherein the sheet substrate of the conductor arrangement is mechanically connected to the carrier substrate of the circuitry and wherein the conductor lines of the conductor arrangement are electrically connected to the conductor lines of the circuitry.

15. A method for fabricating an elongate conductor arrangement, the method comprising:
providing an elongate sheet substrate comprising a support layer consisting of a LCP material,
arranging at least two conductor lines along a surface of the sheet substrate, twisting the sheet substrate around a longitudinal axis of the sheet substrate into a spiral configuration while the sheet substrate being softened due to being subjected to an elevated temperature, and
reducing the temperature while the sheet substrate being in the spiral configuration.
